(19) United States
(12) Patent Application Publication
(10) Pub. No.: US 2025/0290104 A1
(43) Pub. Date: Sep. 18, 2025

(54) METHOD FOR CONSTRUCTION OF CAPROIC ACID-PRODUCING MICROBIAL COMMUNITY AND USE THEREOF

(71) Applicant: SEOUL NATIONAL UNIVERSITY R&DB FOUNDATION, Seoul (KR)

(72) Inventors: Kyoungphile NAM, Seoul (KR); Byung-Chul KIM, Seongnam-si, Gyeonggi-do (KR)

(21) Appl. No.: 18/572,545

(22) PCT Filed: Jan. 27, 2022
(86) PCT No.: PCT/KR2022/001487

§ 371 Date: Dec. 20, 2023

(30) Foreign Application Priority Data
Jun. 21, 2021 (KR) 10-2021-0080365

(51) Int. Cl. C12P 7/40; C12N 1/20

(52) U.S. Cl. CPC C12P 7/40; C12N 1/20

(57) ABSTRACT

This relates to a method of preparing a caproate-producing microbiome and a caproate production method using the prepared microbiome, in which, according to an aspect of the method of preparing a caproate-producing microbiome, the caproate-producing microbiome may be prepared by making a caproate-producing strain dominant in an anaerobic microbiome, and the microbiome has an advantage in that caproate may be produced with high efficiency.

### CROSS REFERENCE TO RELATED APPLICATIONS

This application is a 371 National phase filing of PCT/KR2022/001487, filed Jan. 27, 2022, which application claims priority to Korean Patent Application No. 10-2021-0080365, filed Jun. 21, 2021, in the Korean Intellectual Property Office, the contents of which are hereby incorporated by reference in their entirety for all purposes.

### SEQUENCE LISTING

The instant application contains a Sequence Listing which has been submitted electronically in ASCII format and is hereby incorporated by reference in its entirety. Said ASCII copy, created on Jan. 12, 2024, is named 110623-1418780_SL.txt and is 941 bytes in size.

### TECHNICAL FIELD

It relates to a method of preparing a caproate-producing microbiome and a caproate production method using the prepared microbiome.

### BACKGROUND ART

In the natural environment, microorganisms rarely exist as a single species, but form a microbial community (microbiome) that form interrelationships with other species of microorganisms, etc., and this characteristic has been identified in almost all microorganisms found in the environment, including wastewater treatment, water purification systems, pathogen survival and antibiotic resistance, polluted environment restoration, and metal corrosion. Since the expression characteristics of microorganisms when forming a community are different from those when existing as a single species, industrial applications of microbiomes are being studied by discovering microbiomes as functional expression units and analyzing the kinetic characteristics of the microorganisms of which they consist, changes in microbial phenotype and activity according to environmental changes, and interrelationships between microbial cells.

Caproate is a carboxylate including six carbons and is colorless and has a cheesy scent, is found in the fat of many animals, and is called hexanoic acid. Caproate is a non-toxic medium-chain fatty acid that is easily absorbed into the body, and thus is widely used as an additive in products such as foods, pharmaceuticals, and cosmetics. Caproate is used as a precursor for the production of hexyl phenols and hexyl derivatives, and the resulting hexyl derivatives are used as food additives for perfumes and scents. Among the hexyl derivatives, hexanol is a compound with high utility value that may be used as a fuel additive in aircraft.

Recently, the production of n-caproate from organic waste resources by using an open culture anaerobic microbiome is receiving much attention due to its high economic benefits and low environmental impact. However, due to the high ecological complexity and genetic diversity of anaerobic microbiomes, the composition of fermentation products is unpredictable and productivity is low.

Therefore, to overcome these limitations, in the present disclosure a platform construction technology was developed to stably produce n-caproate using organic waste resources as raw materials.

### DISCLOSURE

### Technical Problem

An aspect provides a method of preparing a caproate-producing microbiome, including a process of inoculating an anaerobic microbiome into a media composition including lactate; and a media composition process of culturing.

Another aspect provides a method of producing caproate, including the process of inoculating an anaerobic microbiome with a media composition including lactate; and the media composition process of culturing.

Another aspect provides a method of producing caproate, including the process of inoculating an organic waste resource including lactate with a caproate-producing microbiome prepared by the method; and the media composition process of culturing.

### Technical Solution

An aspect is to provide a method of preparing a caproate-producing microbiome, including a process of inoculating an anaerobic microbiome with a media composition including lactate; and a media composition process of culturing.

As used herein, the term “caproate” refers to a saturated carboxylate that has six carbons, also referred to as “hexanoic acid” or “caproic acid”, which may be represented by the chemical formula CH₃(CH₂)₄COOH. The caproate has high economic potential as a platform chemical.

As used herein, the term “culture” may refer to growing microorganisms under suitable artificially controlled environmental conditions, which may include a fermentation process.

The anaerobic microbiome refers to a collection or colony of anaerobic microorganisms as obtained from anaerobic digestive sludge.

As used herein, the term “anaerobic microorganisms” refers to oxygen-free microorganisms that do not require oxygen, which may be generally categorized as facultative anaerobes, aerotolerant anaerobes, and obligate anaerobes. Facultative anaerobes are microorganisms that may grow with or without oxygen, aerotolerant anaerobes are microorganisms that may live in the presence of oxygen but do not utilize oxygen, and obligate anaerobes are microorganisms that cannot grow in the presence of free oxygen and therefore may only live in the absence of oxygen.

The caproate-producing microbiome may include a caproate-producing strain, and specifically, may refer to a microbiome that is dominated by a caproate-producing strain relative to other strains and may be utilized to effectively produce caproate.

The caproate-producing strain may include one or more selected from the group consisting of Caproiciproducens genus, including Ruminococcacea spp., Ruminococcaceae bacterium CPB6, Clostridium kluyveri, Megasphaera elsdenii, Megasphaera hexanoica, Clostridium carboxidivorans, and Caproiciproducens galactitolivorans, etc.

The process of culturing may include a process of increasing the ratio of caproate-producing strains in the inoculated anaerobic microbiome and/or decreasing the ratio of strains capable of inhibiting caproate production, and may specifically include a process of dominating the caproate-producing strains in the anaerobic microbiome and/or culling the strains capable of inhibiting caproate production.

The strains capable of inhibiting caproate production may include strains capable of inducing a competitive reaction of caproate production in the process of culturing, and may specifically include propionate-producing strains.

The propionate-producing strain may include one or more selected from the group consisting of Propionibacterium genus, including Propionibacterium freudenreichii, Propionibacterium acidipropionici, Propionibacterium jensenii, Propionibacterium thoenii, etc., Veillonella gazogenes, Veillonella criceti, Veillonella alcalescens, Veillonella parvula, Clostridium homopropionicum, Bacteroides spp. and Fusobacterium necrophorum.

The media composition may include components necessary to dominate caproate-producing strains and/or to cull propionate producing strains within the anaerobic microbiome.

The media composition may include lactate as the electron donor, and specifically, the lactate may be included in the media composition at a concentration of 10 mM to 150 mM, 20 mM to 150 mM, 30 mM to 150 mM, 40 mM to 150 mM, 50 mM to 150 mM, 70 mM to 150 mM, 10 mM to 130 mM, 20 mM to 130 mM, 30 mM to 130 mM, 40 mM to 130 mM, 50 mM to 130 mM, 70 mM to 130 mM, 10 mM to 100 mM, 20 mM to 100 mM, 30 mM to 100 mM, 40 mM to 100 mM, 50 mM to 100 mM, or 70 mM to 100 mM.

The media composition may additionally include butyrate as an electron acceptor, and specifically, the butyrate may be included in the media composition at a concentration of 1 mM to 50 mM, 1 mM to 40 mM, 1 mM to 30 mM, 1 mM to 20 mM, 3 mM to 50 mM, 3 mM to 40 mM, 3 mM to 30 mM, 3 mM to 20 mM, 5 mM to 50 mM, 5 mM to 40 mM, 5 mM to 30 mM, or 5 mM to 20 mM.

The media composition may include lactate and butyrate, and specifically, the lactate and butyrate may be included in the media composition at a ratio of 1:1 to 20:1, 2:1 to 20:1, 4:1 to 20:1, 8:1 to 20:1, 1:1 to 15:1, 2:1 to 15:1, 4:1 to 15:1, 8:1 to 15:1, 1:1 to 10:1, 2:1 to 10:1, 4:1 to 10:1, 8:1 to 10:1, 1:1 to 8:1, 2:1 to 8:1, 4:1 to 8:1, 1:1 to 6:1, 2:1 to 6:1, or 4:1 to 6:1.

In the process of culturing, the headspace gas may include hydrogen, specifically, may include 5%.

The process of culturing may additionally include a process of removing carbon dioxide from the headspace, specifically removing carbon dioxide produced by the conversion of lactate to acetyl-CoA.

In the process of culturing, the pH of the media composition may be from 5 to 6.

The process of culturing may be culturing for about 100 days or less, for example, may be culturing for 1 to 100 days, 1 to 90 days, 1 to 80 days, 1 to 70 days, 1 to 60 days, 1 to 50 days, 5 to 100 days, 5 to 90 days, 5 to 80 days, 5 to 70 days, 5 to 60 days, 5 to 50 days, 10 to 100 days, 10 to 90 days, 10 to 80 days, 10 to 70 days, 10 to 60 days, 10 to 50 days, 15 to 100 days, 15 to 90 days, 15 to 80 days, 15 to 70 days, 15 to 60 days, 15 to 50 days, 20 to 100 days, 20 to 90 days, 20 to 80 days, 20 to 70 days, 20 to 60 days, 20 to 50 days, 25 to 100 days, 25 to 90 days, 25 to 80 days, 25 to 70 days, 25 to 60 days, 25 to 50 days, 30 to 100 days, 30 to 90 days, 30 to 80 days, 30 to 70 days, 30 to 60 days, or 30 to 50 days.

The process of culturing may be culturing in a batch culture, a continuous culture, a semi-continuous culture, or a combination thereof, and may specifically include performing a batch culture alone, culturing the microbiome obtained after the batch culture in a continuous culture or semi-continuous culture method, or culturing in a sequence of batch culture, semi-continuous culture, and continuous culture.

As used herein, the term “batch culture” refers to a culture method in which the media is changed again after the culture is complete, in other words, the media is initially filled once and no further nutrients are added or removed until the end of the culture. It is assumed that the batch incubator is equipped with a stirrer so that the composition of the contents is uniform.

If the process of culturing is performed in a batch culture, it may be culturing in a sequential batch reactor. The sequential batch culture refers to performing the batch culture several times in a row, specifically, determining that the batch culture has ended when the lactate included in the media composition is depleted and repeating the batch culture again in a fresh media composition. the sequential batch culture may be repeated from 1 time to 10 times, from 1 time to 7 times, from 1 time to 5 times, from 2 times to 4 times, or from 3 times to 7 times.

As used herein, the term “continuous culture” refers to a culture method in which a fresh media is continuously supplied to an incubator (fermenter) at a constant rate and an equal amount of culture liquid is continuously discharged so that the liquid volume in the incubator is always constant, which has the advantage that the culture conditions are always constant, but has the disadvantage that there is a risk of contamination.

The semi-continuous culture is a modified method of the continuous culture, in which the parts of supplying fresh media and discharging the same amount of culture media are the same, but the difference is that the supply and discharge process is not carried out continuously, but at a certain time. Specifically, in the present disclosure, the culture media is discharged for 15 minutes every 24 hours, and after 10 minutes fresh media is injected for 15 minutes.

The semi-continuous culture may be carried out at a temperature condition of 20 to 30° C., and specifically may be carried out at a temperature condition of 20 to 28° C., 20 to 26° C., 22 to 28° C., 22 to 26° C., 24 to 28° C., or 24 to 26° C.

As used herein, the continuous culture or semi-continuous culture is used interchangeably with the term “continuously fed culture” or “semi-continuously fed culture”.

The continuous culture or semi-continuous culture may be performed in an anaerobic membrane bioreactor (AnMBR).

Another aspect is to provide a method of producing caproate, including the processes of inoculating an anaerobic microbiome in a media composition including lactate; and culturing the media composition. The same aspects as described above apply equally to the method.

The process of culturing may be performed under the same conditions as the process of culturing in the method of preparing a caproate-producing microbiome.

The process of culturing may be culturing in a batch culture, a continuous culture, a semi-continuous culture, or a combination thereof, and specifically may include performing a batch culture alone, or culturing the microbiome obtained after the batch culture in a continuous culture or semi-continuous culture method.

The method may additionally include the process of recovering caproate from the culture.

As used herein, the term “culture” refers to a material, including a media, in which microorganisms are growing or have been grown under moderately artificially controlled environmental conditions. In a narrow sense, a culture does not include live microorganisms, but may include live microorganisms in a broader sense. The culture may include, in addition to the components of a media composed for culturing microorganisms, various substances discharged into the media by the microorganisms during growth, and may specifically include the target substance caproate.

The process of recovering the caproate may include methods known in the art, such as centrifugation, filtration, anion exchange chromatography, crystallization, and HPLC, etc.

Another aspect is to provide a method of producing caproate, including the process of inoculating an organic waste resource including lactate with a caproate-producing microbiome prepared by the method; and the process of culturing the media composition. The same aspects as described above also apply to the above method.

The organic waste resource is rich in lactate or lactose, and may specifically be milk processing wastewater.

If the organic waste resource contains lactose, a fermentation process may be added to convert the lactose to lactate.

### Advantageous Effects

According to a method of preparing a caproate-producing microbiome according to an aspect, a caproate-producing microbiome may be prepared by dominating a caproate-producing strain in an anaerobic microbiome, and the microbiome has the advantage of being able to produce caproate with high efficiency.

### DESCRIPTION OF DRAWINGS

FIG. 1 is a schematic view illustrating a design-build-test-learn engineering framework.

FIG. 2 is a diagram illustrating a change of even-carbon number carboxylate concentrations during a 1st DBTL cycle operation period (on a lactate equivalent basis, 1, 2 and 3 lactate molecules are required to produce 1 molecule of acetate, n-butyrate and n-caproate, respectively).

FIG. 3 is a diagram illustrating a concentration of carboxylates produced after a 2nd DBTL cycle operation period (on a lactate equivalent basis, 1, 1, 2 and 3 lactate molecules are required to produce 1 molecule of acetate, propionate, n-butyrate and n-caproate, respectively).

FIG. 4 is a diagram illustrating a change in H₂ composition of a headspace gas during the 2nd DBTL cycle operation period.

FIG. 5 is a diagram illustrating an experimental setup for a 3rd DBTL cycle.

FIG. 6 is a diagram illustrating selectivity of carboxylates produced after the 3rd DBTL cycle operation period (on a lactate equivalent basis, 1, 1, 2, 2, 2, and 3 lactate molecules are required to produce 1 molecule of acetate, propionate, n-butyrate, n-valerate, and n-caproate, respectively).

FIG. 7 is a diagram illustrating the relative abundance of bacteria present in a microbiome formed after operation with an R5 experimental setup of the 3rd DBTL cycle.

FIG. 8 is a diagram illustrating the concentration of carboxylates produced according to each iteration of the R5 experimental setup in repeated batch cultures.

FIG. 9 is a diagram illustrating a change in concentration of substrate and carboxylate during operation with a R5-OLR_{increased} experimental setup.

FIG. 10 is a diagram illustrating the relative abundance of bacteria in a biomass sample at the beginning of a culture (Ini), and at the first (1st) and at the fifth (5th) replicate, in a batch culture repeated with the R5 experimental setup. DNA and RNA denote 16S rRNA sequencing and 16S rRNA gene amplicon sequencing, respectively.

FIG. 11 is a schematic view illustrating an anaerobic membrane bioreactor. HFM, DP, and PP stand for hollow-fiber membrane module, diaphragm pump, and peristaltic pump, respectively.

FIG. 12 is a diagram illustrating carboxylate concentrations in effluent according to a cycle in a semi-continuously fed culture.

FIG. 13 is a diagram illustrating selectivity of carboxylates produced under various culture conditions.

FIG. 14 is a diagram illustrating the relative abundance of bacteria in each biomass sample at a 7th, 29th, and 50th cycle during a semi-continuously fed AnMBR operation. The samples were obtained from media, a reactor, and a membrane.

FIG. 15 is a diagram illustrating the concentration of carboxylates in effluent according to an operation period in a culture under AnMBR_StoC conditions.

FIG. 16 is a diagram illustrating the relative abundance of bacteria in each biomass sample at the beginning, day 7, and day 13 of a culture operated under AnMBR_StoC conditions. The samples were obtained from media, and a reactor.

FIG. 17 is a diagram illustrating the concentration of carboxylates in effluent according to an operation period in a culture under AnMBR_cont conditions.

FIG. 18 is a diagram illustrating results of a carbon mass balance analysis in effluent and influent according to an operation period from day 25 to day 32 in a culture under AnMBR_cont conditions.

FIG. 19 is a diagram illustrating the relative abundance of bacteria in a sample on day 0, day 7, and day 40 of operation in a culture under AnMBR_cont conditions.

FIG. 20 is a diagram illustrating changes in concentration of substrates (lactose, glucose, fructose, galactose) and products (lactate, formate, acetate) during a lactic acid fermentation process.

FIG. 21 is a diagram illustrating concentration changes of substrates (lactate, galactose, lactose) and products (formate, acetate, n-butyrate, n-caproate, propionate, n-valerate) during a batch reactor operation process.

### MODE FOR INVENTION

The following embodiments will describe in more detail. However, these embodiments are for illustrative purposes only and the scope of the present disclosure is not limited to these embodiments.

### Example 1: Obtaining Anaerobic Digestion Sludge and Inoculum

The anaerobic digestion sludge used in the embodiments of the present disclosure was obtained from the waste activated sludge anaerobic digestive system of the Seoul Junglang Sewage Treatment Plant. The sludge was filtered through a 0.5 mm sieve to remove impurities, and stored at 4° C. until use.

The sludge was diluted to 1% (v/v) in a modified basal media including trace metal solution, vitamin solution, 1.25 g/L of yeast extract and 19.5 g/L of MES [2-(N-morpholino)ethanesulfonic acid] sodium salt. Glucose at 2 g COD/L was added as a carbon source and the pH was adjusted to 5.6 using HCl. 500 mL of the diluted sludge was dispensed into a 1 L bottle, purged with N₂ gas to produce anaerobic conditions, and cultured at 35° C. in a constant temperature darkroom for 24 hours. The bottle was continuously stirred using a magnetic bar, and a Tedlar bag (Supelco, Pennsylvania, USA) was attached to keep the headspace pressure at atmospheric pressure. The anaerobic cultures obtained by the culture were centrifuged at 3000×g for 10 minutes and the supernatant was removed. The cell pellet was gently resuspended in modified basal media purged with N₂ gas to obtain inoculum.

### Example 2: Establishing Optimal Conditions for Forming n-Caproate Producing Microbiome

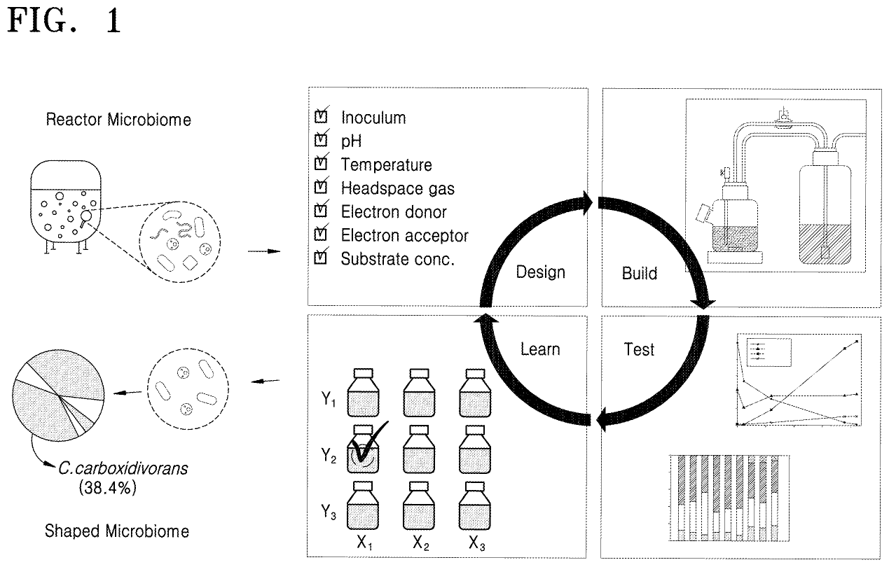

In the present disclosure, experiments were conducted based on a design-build-test-learn engineering framework to establish optimal culture conditions for shaping an n-caproate producing microbiome (FIG. 1). Specifically, the process was performed by controlling the type and concentration of substrates and headspace CO₂ and H₂ partial pressures. Among various experimental conditions, the condition showing the best n-caproate productivity (in other words, yield and rate) was considered as the optimal shaping condition of the cycle.

Meanwhile, pH and CO₂ partial pressure were selected as the main operating parameters of the batch reactor for forming the n-caproate producing microbiome from anaerobic digestion sludge. Specifically, methane production may consume acetate, one of the starting compounds of the chain extension reaction, by different pathways. Therefore, since methane production should be inhibited during the chain extension reaction, all experiments performed in the embodiments of the present disclosure were set to an initial pH value of 5.5 to inhibit the activity of methanogens. In addition, the initial CO₂ partial pressure was set to 0.2 atm in the present disclosure.

Apart from pH and CO₂ partial pressure, H₂ partial pressure and the type and concentration of electron donors and electron acceptors are important for forming the n-caproate producing microbiome. Therefore, the parameters were determined based on a DBTL framework through the following process.

### 2.1: 1st DBTL Cycle

A 1st DBTL cycle was designed to test the effect of the type of electron donor and the presence of headspace H₂ on forming the n-caproate producing microbiome. Tests were performed with ethanol and lactate, which are representative fermentation products among various electron donors for chain extension reactions.

Specifically, 50 mL of the inoculum obtained in Example 1 above was dispensed into a 285 mL bottle, purged with a mixture of gases (N₂ 75%, CO₂ 20% and H₂ 5%; or N₂ 80% and CO₂ 20%) and the bottle was sealed. Eight experimental groups were set up, labeled L-O, L-X, E-O, and E-X, respectively. L and E refers to electron donors lactate (L) and ethanol (E) used in the cycle, respectively, and O and X refers to presence (O) or absence (X) of H₂ in the mixed gas used for purge, respectively. Each experimental group was performed in three repetitions and stirred in a shaking incubator (35° C., 120 rpm). Headspace gas and liquid fractions were sampled and analyzed every 24 hours.

Composition of headspace gases (CO₂, N₂, CH₄, H₂) was measured by gas chromatography. Gas samples were separated using a Carboxen 1000 column (Sigma-Aldrich) and detected using a thermal conductivity detector. Concentrations of carboxylate and L-lactate were measured after filtering the liquid samples using a 0.45 μm syringe GHP filter. Concentration of carboxylates (in other words, SCC and MCC) in the liquid samples was measured by gas chromatography (Agilent, California, USA). Analytes were separated using an HP-INNOWax GC column (Agilent, California, USA) and detected using a flame ionization detector. Concentration of L-lactate was determined using an L-lactate kit (Megazyme, Bray, Ireland).

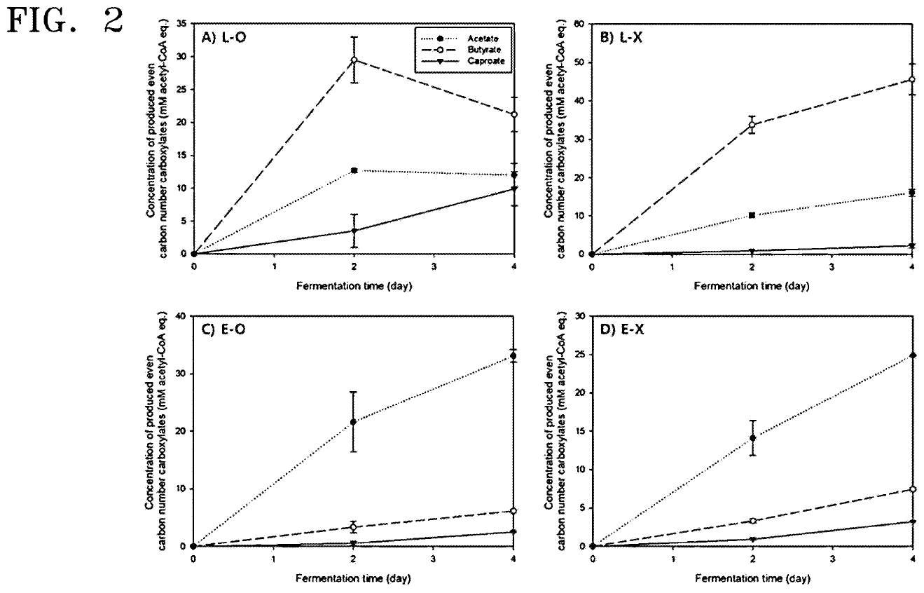

First, L-lactate (50 mM) and ethanol were added to the inoculum, which was an adapted anaerobic digestion sludge under slightly acidic conditions, and it was found that n-butyrate was the dominant fermentation product when L-lactate was added as an electron donor (in other words, 10.6 Mm and 15.2 mM of n-butyrate were produced in L-O and L-X, respectively), while acetate production was dominant when most of the ethanol was added (in other words, 33.1 nM and 24.9 mM of acetate were prepared in E-O and E-X, respectively) (FIG. 2). Furthermore, after 4 days of fermentation, the concentrations of n-caproate were 3.3 mM, 0.75 mM, 0.83 mM, and 1.07 mM in L-O, L-X, E-O, and E-X, respectively, and the selectivity values of n-caproate on an acetyl-CoA equivalent basis were 19.8%, 4.5%, 5.0%, and 6.4%, respectively (FIG. 2). Based on the results, it may be seen that it is advantageous to use lactate as an electron donor to effectively form the n-caproate producing microbiome.

Next, it was confirmed if supplying H₂ to the headspace during culture was advantageous for n-caproate production. Specifically, H₂-supplied L-O was 5 times more productive of n-caproate than L-X. Furthermore, it was also confirmed that when the H₂ partial pressure is lower than 60 Pa, the reducing power of NADH may be used to reduce H+ to produce H₂, rather than a chain extension reaction in which acetyl-CoA binds to an existing acyl-CoA chain (in other words, acetyl-CoA or butynyl-CoA) (FIG. 2). Based on the results, it may be seen that it is efficient to set the initial partial pressure of H₂ at 5%.

Taking the above results together, may be seen that in order to establish an n-caproate producing microbiome, it is advantageous to utilize lactate as an electron donor and supply H₂ to the headspace.

### 2.2: 2nd DBTL Cycle

A 2nd DBTL cycle was designed to test the effect of electron acceptors on forming the n-caproate producing microbiome.

Specifically, 50 mL of the inoculum obtained in Example 1 above was dispensed into a 285 mL bottle, purged with a mixture of gases (N₂ 75%, CO₂ 20%, H₂ 5%) and the bottle was sealed. Next, three experimental groups were set up (L50, L50-A10, and L50-B10). The L50 contained 50 mM L-lactate as an electron donor and did not include an electron acceptor, while a media of L50-A10 and L50-B10 contained 10 mM of acetate or n-butyrate, respectively, along with 50 mM of L-lactate. Each experimental group was performed in three repetitions and stirred in a shaking incubator (35° C., 120 rpm). Headspace gas composition was analyzed every 24 hours, and the liquid fraction was sampled after 4 days of fermentation.

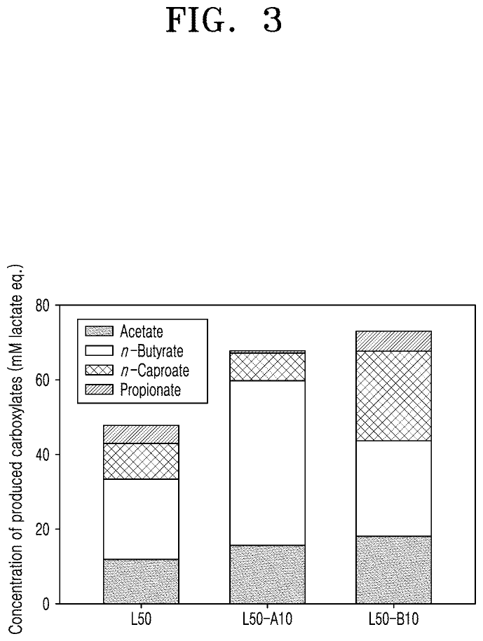

As a result, in the absence of the electron acceptor (L50), 3.3 mM of n-caproate (19.8% caproate selectivity) was produced, while n-butyrate was dominantly produced (10.6 mM; 42.4% n-butyrate selectivity). When n-butyrate was used as the electron acceptor, the production of n-caproate was highest at 7.9 mM (33.9% n-caproate selectivity). Production of propionate was negligible (in other words, <0.5 mM, <1% on a lactate equivalent basis) in L50-A10, but accounted for 9.8% and 7.4% (on a lactate equivalent basis) of the carboxylates produced in L50 and L50-B10, respectively. In addition, the fermentation of n-butyrate was accelerated in L50-A10, showing n-butyrate selectivity of 74.0% (FIG. 3). Based on the results, it was confirmed that it is advantageous to utilize butyrate as an electron acceptor to efficiently form the n-caproate producing microbiome.

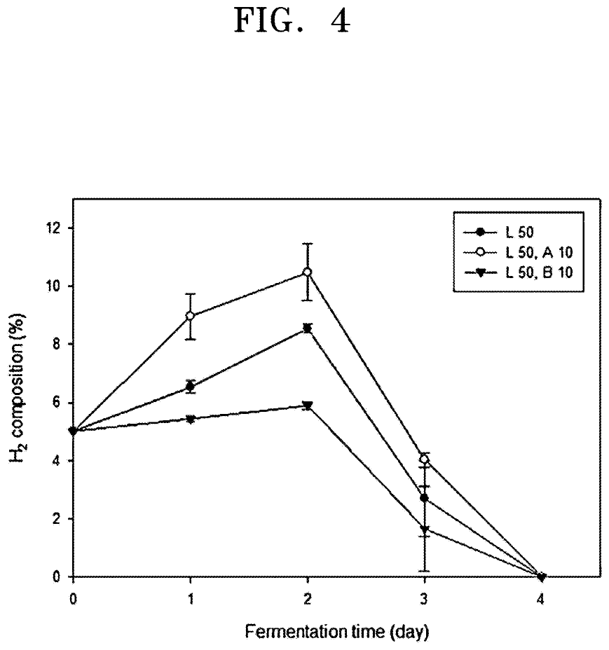

Furthermore, the total concentration of carboxylates (acetate, propionate, n-butyrate and n-caproate) produced in L50, L50-A10 and L50-B10 were 47.8 mM, 67.7 mM and 73.0 mM (lactate equivalent), respectively, which were higher than the initial concentration (in other words, 50 mM, 60 mM and 70 mM, respectively). Therefore, to confirm the involvement of headspace H2 in the production of carboxylate in the reaction, the gas composition of the headspace was monitored during the reaction period. As a result, the H₂ partial pressure was found to increase during the first 2 days of the reaction and then decrease, confirming that the headspace H₂ was completely depleted by day 4 (FIG. 4). Based on the results, it may be seen that chemolithotrophic homoacetogens such as Clostridium scatologenes, Clostridium autoethanogenum, and Clostridium ljungdahli may be present in the anaerobic digestive sludge as an inoculum.

Chemolithotrophic homoacetogens may produce acetate using H₂ and CO₂ as reactants, and n-caproate is difficult to produce when H₂ is depleted. In addition, when a chain extension bacteria use lactate to produce caproate, CO₂ may be produced as lactate is converted to acetyl-CoA. Therefore, it may be seen that CO₂ must be continuously removed from the headspace gas to inhibit chemolithotrophic homoacetogens activity and maintain H₂.

Taking the above results together, it may be seen that in order to establish an n-caproate producing microbiome, it is advantageous to use butyrate as an electron acceptor and continuously remove CO₂.

### 2.3: 3rd DBTL cycle

A 3rd DBTL cycle was designed to identify the effect of continuous headspace CO₂ removal on the chain extension reaction and to determine the optimal concentration of an electron donor (L-lactate) and an electron acceptor (n-butyrate) for forming the n-caproate producing microbiome.

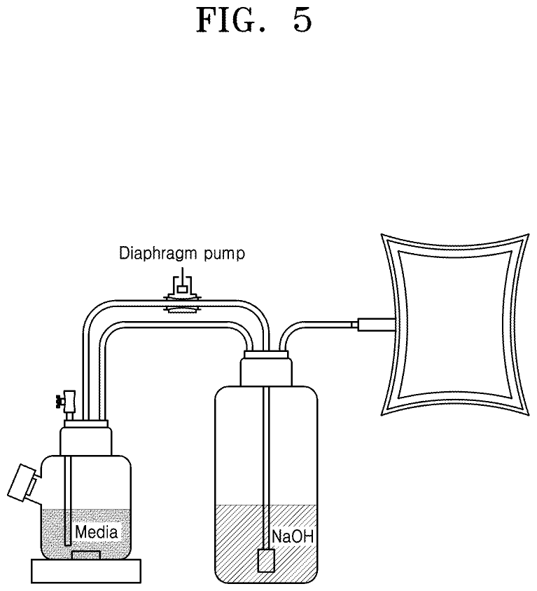

Specifically, each experimental setup consisted of an inoculum bottle (250 ml bottle with two inlets), a bottle of NaOH aqueous solution (1 L bottle), and a diaphragm pump (Boxer, London, UK) (FIG. 5). The inoculum bottle was dispensed with 50 mL of inoculum, purged with N₂ gas, and the bottle sealed. A NaOH solution was used as an absorbent to continuously remove headspace CO₂. The NaOH solution bottle was dispensed with 500 mL of 1 M NaOH solution and purged with N2 gas. The two screw fittings on each bottle were connected to the other bottle using polyurethane tubing. The remaining screw fittings on the inoculum bottle and the NaOH solution bottle were used as sampling and pressure control ports, respectively. During the batch operation period, the headspace gas in the inoculum bottle was continuously pumped into the NaOH solution using a diaphragm pump (Boxer, London, UK). H₂ gas was injected into the headspace to set the initial H₂ partial pressure to 0.05 atm. Under the 2nd DBTL cycle condition, 2 repetitions were performed with the following nine types of experimental setups. L-lactate (electron donor) at 25 mM (R1 to R3), 50 mM (R4 to R6), and 100 mM (R7 to R9) and n-butyrate (electron acceptor) at 5 mM (R1, R4, and R7), 10 mM (R2, R5, and R7), and 20 mM (R3, R6, and R9) were injected, respectively (Table 1).

TABLE 1

n-Butyrate

| | | 5 mM | 10 mM | 20 mM |
|---|---|---|---|---|
| L-lactate | 25 mM | R1 | R2 | R3 |
| | 50 mM | R4 | R5 | R6 |
| | 100 mM | R7 | R8 | R9 |

A magnetic bar was used to keep the bottle stirred, and a Tedlar bag (Supelco, Pennsylvania, USA) was attached to keep the headspace pressure at atmospheric pressure. The liquid fraction (0.5 mL) was sampled every 6 hours, and the batch operation was stopped when no L-lactate was detected in the liquid sample.

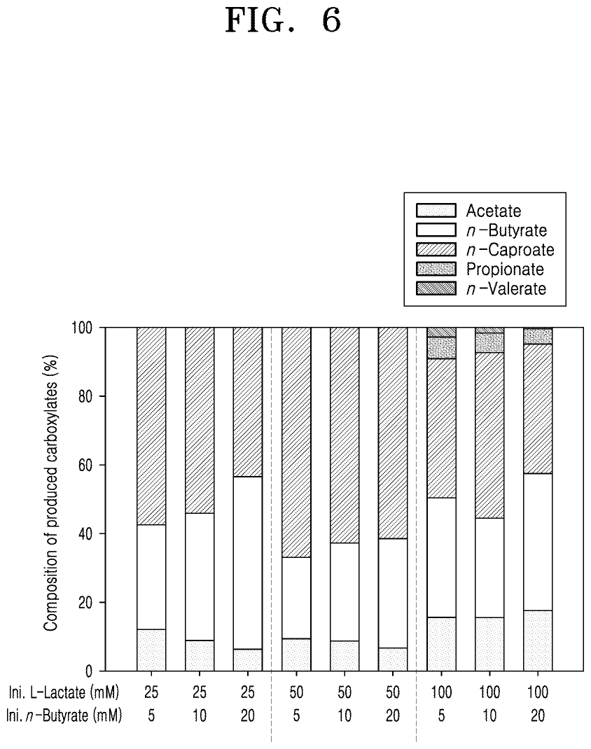

First, the effect of continuous CO₂ removal on n-caproate productivity was confirmed. As a result, it was confirmed that no CO₂ was detected in the headspace during the reaction, which significantly increased the selectivity of n-caproate. Specifically, when the anaerobic digestive sludge was treated with 50 mM L-lactate and 10 mM n-butyrate (R5 of FIG. 6) while continuously removing CO₂, the selectivity of n-caproate was 66.7% on a lactate equivalent basis, while the selectivity of n-caproate was 33.9% when cultured under the same conditions without CO₂ removal (L50-B10 of FIG. 3). Therefore, based on the experimental results, it may be seen that n-caproate productivity may be promoted by continuously removing CO₂ from the culture conditions to inhibit the progress of the competitive reaction.

Furthermore, these results are contrary to previous studies that have confirmed that caproate production efficiency may be increased by the injection of carbon dioxide during the production of caproate using ethanol as an electron donor. Therefore, it may be seen that the condition of continuously removing carbon dioxide to improve n-caproate productivity is a new condition identified in the present disclosure. Next, various concentrations of L-lactate and n-butyrate (in other words, 25 mM, 50 mM, 100 mM and 5 mM, 10 mM, 20 mM, respectively) were treated to determine the optimal concentration of an electron donor and an electron acceptor. As a result, the consumption rate of L-lactate in the case of 100 mM L-lactate treatment (R7, R8, and R9 in Table 1) was slower than in the case of 25 mM or 50 mM L-lactate treatment, and the concentration of n-caproate produced was about 14.1 to 20.7 mM (Table 2).

TABLE 2

| | Initial | Initial | Final | | Time |
|---|---|---|---|---|---|
| | L- | n- | n- | | required |
| | lactate | butyrate | caproate | n- | to |
| | concen- | concen- | concen- | Caproate | deplete |
| Experiment | tration | tration | tration | selectivity | L-lactate |
| Setup | (mM) | (mM) | (mM) | (%) | (hous) |
| R1 | 25 | 5 | 6.4 | 55.0 | 16 |
| R2 | | 10 | 7.7 | 51.6 | 16 |
| R3 | | 20 | 9.3 | 43.2 | 16 |
| R4 | 50 | 5 | 13.7 | 68.6 | 29 |
| R5 | | 10 | 15.6 | 66.7 | 29 |
| R6 | | 20 | 18.2 | 60.8 | 40 |
| R7 | 100 | 5 | 20.7 | 56.3 | 168¹⁾ |
| R8 | | 10 | 20.4 | 51.1 | 168¹⁾ |
| R9 | | 20 | 14.1 | 30.3 | 168¹⁾ |

¹⁾L-lactate still remains in the bottle.

### 1) L-Lactate Still Remains in the Bottle

In addition, odd-carbon number carboxylates (propionate and n-valerate) were detected (FIG. 6). Lactyl-CoA, a precursor of propionate, is known to be produced under conditions of excess L-lactate in pure culture. Therefore, the results indicate that 100 mM of L-lactate is excessive for forming the n-caproate producing microbiome, and that high lactate residual concentrations may induce propionate production rather than n-caproate.

Furthermore, when 25 mM or 50 mM of L-lactate was treated with 5 mM, 10 mM, or 20 mM of n-butyrate (R1-R6), the L-lactate was completely consumed after 16 hours or 40 hours, respectively (Table 2). Specifically, when the initial concentration of L-lactate was 50 mM, the n-caproate selectivity were 68.6%, 66.7%, and 60. 8% when treated with 5 mM, 10 mM, and 20 mM n-butyrate, respectively (R4, R5, and R6 in Table 2 and FIG. 6), and when the initial L-lactate concentration was 25 mM, the n-caproate selectivity were 55.0%, 51.6%, and 43.2% when treated with 5 mM, 10 mM, and 20 mM n-butyrate, respectively (R1, R2, and R3 in Table 2 and FIG. 6). Therefore, it may be seen that utilizing 50 mM L-lactate as an electron donor may efficiently form the n-caproate producing microbiome.

Next, the specificity (Speⁱ) of carboxylate (i) was calculated using the following formula.

$$S p {e}^{i} = {γ}^{i} \times {C}^{i} / {\sum }_{j} ( {γ}^{j} \times {C}^{j} )$$

[rⁱ (mole carbon/mole carboxylate i) refers to the number of carbon atoms included in the carboxylate i, and Cⁱ refers to a concentration of carboxylate i in an effluent].

As a result, the final concentration of n-caproate increased with increasing concentrations of n-butyrate, but the selectivity and specificity of n-caproate decreased (Table 2 and FIG. 6). Specifically, when 20 mM n-butyrate was injected while treating 50 mM L-lactate (R6), the consumption rate of L-lactate was significantly slower than when treated with 5 mM and 10 mM n-butyrate. Considering the productivity of n-caproate, it was higher when treated with 10 mM n-butyrate (3.31 gCOD/L/day) than with 5 mM n-butyrate (2.90 gCOD/L/day) or 20 mM n-butyrate (2.80 gCOD/L/day) (Table 2). Therefore, it may be seen that 10 mM L-butyrate as an electron acceptor may efficiently form an n-caproate producing microbiome.

Taking the above results together, it may be seen that it is advantageous to use 50 mM of L-lactate as an electron donor and 10 mM of n-butyrate as an electron acceptor to establish an n-caproate producing microbiome, and in addition to the concentrations, the concentration ratio of L-lactate and n-butyrate is also an important factor.

### 2.4: Microbial Composition Analysis of Formed n-Caproate Producing Microbiome

To confirm that the n-caproate producing microbiome could be selectively shaped under a R5 condition of a 3rd DBTL cycle, an optimal condition identified in the embodiments, microbiome analysis was performed using 16S rRNA sequence analysis.

Specifically, an inoculum was prepared and operated in a R5 experimental setup of the 3rd DBTL cycle and liquid samples were collected. The liquid samples were centrifuged at 3000×g for 10 minutes and cell pellets were collected for subsequent analysis. 16S rRNA sequencing (Macrogen Inc., Seoul, Korea) was performed to analyze dynamic changes in microbiome composition. Total RNA was extracted using TRIzol (Life Technologies, New York, USA) following the manufacturer's instructions. For the reverse transcription reaction, 1 μg of total RNA was used to obtain cDNA with SuperScriptII Reverse Transcriptase (Life Technologies GmbH, Darmstadt, Germany). The prepared cDNA sample was amplified using primer 341F (5′-CCTACGGGNGGCWGCAG-3′, SEQ ID NO: 1)-806R (5′-GACTACHVGGGTATCTAATCC-3′, SEQ ID NO: 2). The constructed 16S rRNA library was sequenced using the Illumina Miseq Platform (Illumina, San Diego, USA). Low-quality sequences were removed using CD-HIT-OTU and sequence clustering with 97% similarity was performed to form species-level operational taxonomic units (OTU). Each representative OTU sequence was matched to the NCBI database.

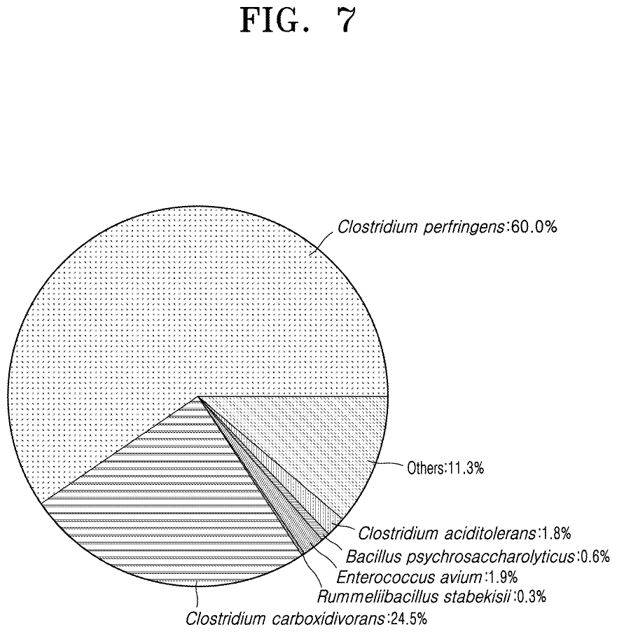

As a result of the above experiment, it was confirmed that the Clostridium carboxidivorans strain was selectively dominant (FIG. 7). The relative occupancy of C. carboxidivorans strains reached 24.5% under R5 experimental conditions. The strain is known to be a solvent-producing, completely anaerobic strain that can fix inorganic carbon to C2 organic carbon (acetate, ethanol) through the Wood-Ljungdahl pathway and condense it with n-caproate, etc. Furthermore, upon analysis of the genome of C. carboxidivorans, two LDHs (L-lactate dehydrogenase) encoding L-lactate dehydrogenase were observed, and it is expected that the strain may utilize L-lactate and condense carbon into n-caproate.

Therefore, it may be seen that culturing an anaerobic microbiome with the optimal condition identified in the embodiment may dominate n-caproate-producing strains including C. carboxidivorans, and that the microbiome including the strain may effectively produce n-caproate.

### Example 3: Establishing Operating Conditions for Sequential Batch Culture to Form n-Caproate Producing Microbiome

### 3.1: Sequential Batch Reactor Operation

To confirm the efficiency of operating a sequential batch reactor as a culture method of shaping and dominating the n-caproate producing microbiome, the following experiments were conducted.

Specifically, the optimal n-caproate producing microbiome shaping conditions identified in Example 2 above were repeatedly cultured in the R5 condition (50 mM L-lactate and 10 mM n-butyrate) of the 3rd DBTL cycle. The concentration of L-lactate was monitored throughout the operation period, and when L-lactate was completely consumed, a 1 mL liquid fraction was sampled to measure the concentration of carboxylates. The anaerobic culture was centrifuged at 3000×g for 10 minutes and transferred to the same media containing 50 mM of L-lactate and 10 mM of n-butyrate. The transferred anaerobic culture was cultured again under the R5 experimental condition. This process was repeated and performed twice.

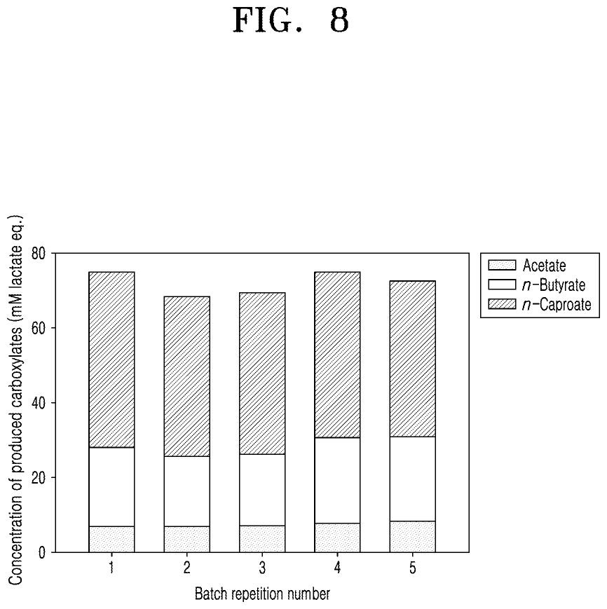

As a result, it took 29 hours for 50 mM of L-lactate to be completely consumed under the R5 condition, and the time required for L-lactate to be completely consumed decreased as the batch culture was repeated, specifically, it was completely consumed in 9 hours or less in the fifth batch culture. After five repeated cultures, the final n-caproate concentration was 13.8 mM to 15.6 mM (57.3% to 62.6% n-caproate specificity) (FIG. 8), and additionally the final concentrations of acetate and n-butyrate were 6.6 mM to 8.1 mM and 9.5 mM to 11.6 mM, respectively. Based on the results, it may be seen that under the optimal microbiome shaping conditions for n-caproate production, by repeating the batch culture operation period, not only carboxylate was stably produced, but also the consumption rate of L-lactate was increased, indicating that the shaping of n-caproate producing microbiome was carried out efficiently.

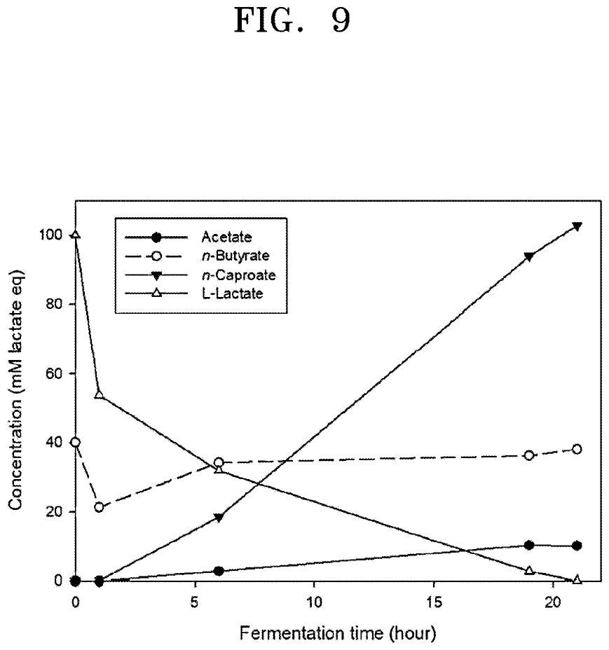

Next, the substrate concentration was increased and the experiment was conducted under the condition of 100 mM L-lactate and 20 mM n-butyrate (R5-OLR_{increased}). As a result, while working with the R5-OLR_{increased} experimental setup, 100 mM of L-lactate was completely consumed after 21 hours of culture. During the culture period, the concentration of n-caproate increased continuously, finally reaching 8.7 g COD/L (34.3 mM) (FIG. 9). Considering the substrate concentrations used for n-caproate production (100 mM of L-lactate and 20 mM of n-butyrate, 140 mM of lactate, on an equivalent basis), the selectivity of n-caproate was calculated to be 72.9% (34 mM of n-caproate, 102 mM of lactate, on a equivalent basis). Meanwhile, the concentration of n-butyrate was maintained during the culture period of the R5-OLR_{increased} experimental setup (20 mM), indicating that no net consumption occurred (FIG. 9). Furthermore, in the experimental setups (in other words, R1, R5, R9, R5-OLR_{increased}) in which L-lactate and n-butyrate were injected in a 5:1 ratio, the change in the concentration of n-butyrate was less than 2 mM and maintained the initial concentration (Table 3).

TABLE 3

| | | Initial | Initial | Final | Change in |
|---|---|---|---|---|---|
| L-lactate | | L-lactate | n-butyrate | n-butyrate | n-butyrate |
| and n- | | concen- | concen- | concen- | concen- |
| butyrate | Experimental | tration | tration | tration | tration |
| ratios | setup | (mM) | (mM) | (mM) | (mM) |
| 20:1 | R7 | 100 | 5 | 26.9 | +21.9 |
| 10:1 | R4 | 50 | 5 | 14.8 | +9.8 |
| | R8 | 100 | 10 | 18.6 | +8.6 |
| 5:1 | R1 | 25 | 5 | 5.1 | +0.1 |
| | R5 | 50 | 10 | 10.7 | +0.7 |
| | R9 | 100 | 20 | 21.8 | +1.8 |
| | R5- | 100 | 20 | 19.0 | −1.0 |
| | OLR_{increased} | | | | |
| 2.5:1 | R2 | 25 | 10 | 8.0 | −2.0 |
| | R6 | 50 | 20 | 14.4 | −5.6 |
| 1.25:1 | R7 | 25 | 20 | 16.5 | −3.5 |

However, the concentration of n-butyrate increased in the experimental condition with a higher ratio of L-lactate to n-butyrate, and the concentration of n-butyrate decreased in the experimental condition with a lower ratio of L-lactate to n-butyrate. Therefore, based on the results, it may be seen that under the experimental conditions of the present disclosure, the production and consumption of n-butyrate occurred simultaneously, and when the initial ratio of L-lactate to n-butyrate was 5:1, the production and consumption reactions were balanced.

### 3.2: Microbial Composition Analysis of Microbiome Formed After Sequential Batch Reaction

To analyze the microbial composition of the n-caproate producing microbiome formed after a sequential batch reaction, the following experiments were conducted.

Specifically, after inoculum preparation, liquid samples were collected after the 1st and 5th operations of the sequential batch reactor (SBR). The liquid samples were centrifuged at 3000×g for 10 minutes and cell pellets were collected for subsequent analysis. 16S rRNA sequencing (Macrogen Inc., Seoul, Korea) was performed to analyze dynamic changes in microbiome composition. Total RNA was extracted using TRIzol (Life Technologies, New York, USA) following the manufacturer's instructions. For the reverse transcription reaction, 1 μg of total RNA was used to obtain cDNA with SuperScriptII Reverse Transcriptase (Life Technologies GmbH, Darmstadt, Germany). The prepared cDNA samples were amplified using primers 341F-806R. The constructed 16S rRNA library was sequenced using the Illumina Miseq Platform (Illumina, San Diego, USA). Low-quality sequences were removed using CD-HIT-OTU and sequence clustering with 97% similarity was performed to form species-level operational taxonomic units (OTU). Each representative OTU sequence was matched to the NCBI database. 16S rRNA gene amplicon sequencing (Macrogen Inc., Seoul, Republic of Korea) was also performed for comparison. The entire DNA was extracted using the PowerMax Soil DNA Isolation Kit (Mo Bio Laboratories, California, USA), amplified, sequenced, and analyzed as described above.

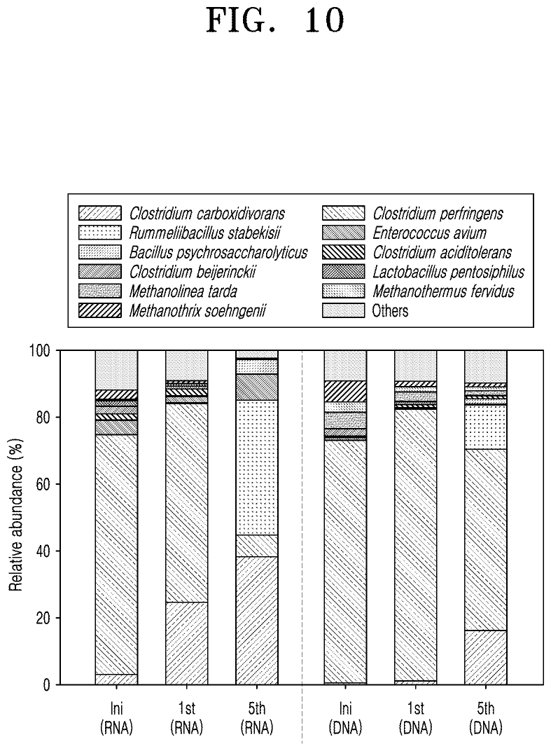

As a result, the relative abundance of C. carboxidivorans reached 38.4% after five repeated batch culture operations with the R5 experimental setup in the 3rd DBTL cycle (FIG. 10). In addition, the relative abundance of Caproiciproducens galactitolivorans, a representative L-lactate-consuming n-caproate-producing bacterium, reached only 0.4% after five repeated batch culture operations with the R5 experimental setup of the 3rd DBTL cycle.

On the other hand, since Caproiciproducens spp. are known to be dominant in the L-lactate supply chain extension microbiome, it was confirmed which of the well-known chain extension bacteria occupy the ecological niche of the shaped microbiome during long-term operation. As a result, it was found that an aerobic bacterium Rummeliibacillus stabekisii increased in abundance (41.0% relative abundance) after five repeated culture operations (FIG. 10). Furthermore, the genome of R. stabekisii was checked in the KEGG database to confirm its function, and it was found that R. stabekisii include chain extension-related genes for n-caproate production.

Next, 16S rRNA gene amplicon sequencing was also performed on the same sample for comparison. As a result, microbial community analysis using RNA reflected the function of the microbiome more clearly than when using DNA. Specifically, the relative abundance of C. carboxidivorans in 16S rRNA sequencing was higher than in 16S rRNA gene amplicon sequencing in both biomass samples collected after the 1st and 5th iteration of repeated operations with the R5 experimental setup of the 3rd DBTL cycle (FIG. 10). Furthermore, the relative abundance of C. carboxidivorans was found to be only 1.12% in the 16S rRNA gene amplicon sequencing of the biomass samples collected after the 1st iteration, despite the active production of n-caproate.

Analysis using RNA is known to be more relevant in describing actual reaction performance than those using DNA, and 16S rRNA sequencing has the advantage of excluding errors due to DNA from inactive or dead cells. Therefore, in order to perform microbiome analysis in a system expected to show dynamic microbial composition changes, using RNA-based tools can yield accurate results.

### Example 4: Establishing Semi-Continuously Fed Anaerobic Membrane Bioreactor Operating Conditions for Forming n-Caproate Producing Microbiome

### 4.1: Operation of a Semi-Continuously Fed Anaerobic Membrane Bioreactor

During operation of the sequential batch culture reactor in Example 3 above, the abundance of C. carboxidivorans increased, but cell immobilization proceeded very slowly due to the slow growth rate. Therefore, as a way to more efficiently culture the n-caproate producing microbiome, the following experiments were conducted to determine the efficiency of operating with an anaerobic membrane bioreactor (AnMBR) designed and operated under semi-continuously fed conditions to mimic SBR.

Specifically, fermentation was carried out in a 2.5 L double-walled reactor. First, a sequential batch reactor (SBR) was operated to obtain shaped microbial communities from anaerobic digestion sludge. After L-lactate was completely depleted, the reactor broth was centrifuged and resuspended in the same media. A circulating water bath was used to set the temperature to 35° C. Shaped microbiome was obtained by repeating the operation three times in the batch reactor.

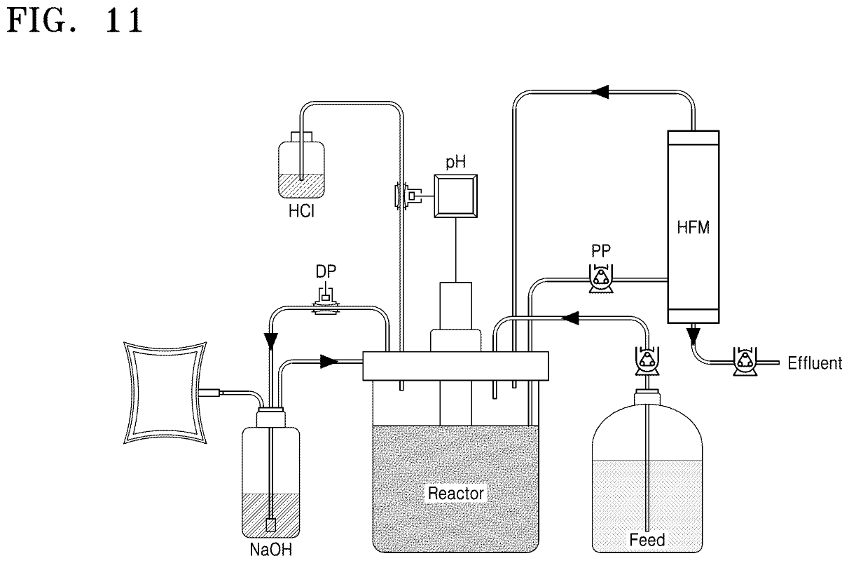

Next, the reactor was operated under a semi-continuous feeding regime. A headspace gas in the reactor was pumped into a 1 M NaOH solution using a gas-lift diaphragm pump. The effluent was filtered using a hollow-fiber hydrophilic membrane module and the biomass was cultured in the reactor. A diagram of the bioreactor setup described above is shown in FIG. 11. The influent media was a modified basic media including trace metal solution, vitamin solution, 1.25 g/L yeast extract, 125 mM L-lactate, and 25 mM n-butyrate. The pH was set to 5.2 using NaOH. During the operation period of the semi-continuously fed anaerobic membrane bioreactor (AnMBR) system, 1 L of effluent was pumped through the hollow-fiber membrane module for 15 minutes every 24 hours. After 10 minutes, 1 L of influent was injected into the reactor for 15 minutes. Using an automatic pH controller connected to a liquid-lift diaphragm pump for adding 3.5% HCl solution, the pH was set to 5.5. The pH was adjusted every 12 hours until the 31st cycle. During cycles 31 to 40, the pH was adjusted continuously. As a result of the above experiment, the SBR operation was first conducted to prepare the inoculum before the AnMBR operation, and the concentration of carboxylates produced during the SBR operation was analyzed. As a result, the concentrations of acetate, n-butyrate, and n-caproate were found to be in the range of 10.18 mM to 11.22 mM, 19.8 mM to 25.2 mM, and 36.18 mM to 43.08 mM, respectively.

Next, during the AnMBR operation, it was found that acetate was present at a certain level, which negatively affected the production of n-caproate in the 2nd DBTL cycle of Example 2. Therefore, to determine if the shaped microbiome could produce n-caproate using acetate, the microbiome grown under the optimal shaping conditions identified in the DBTL cycle was supplied 50 mM of L-lactate and 10 mM of acetate and cultured with the experimental setup used in the 3rd DBTL cycle. As a result, the concentrations of acetate, n-butyrate, and n-caproate were 11.28 mM, 8.42 mM, and 8.78 mM, respectively, and the selectivity of n-caproate was 43.9%, which was 3.5 times higher than the result of L50-A10 in the 2nd DBTL cycle. Therefore, it may be seen that the shaped n-caproate producing microbiome may produce n-caproate even under acetate conditions.

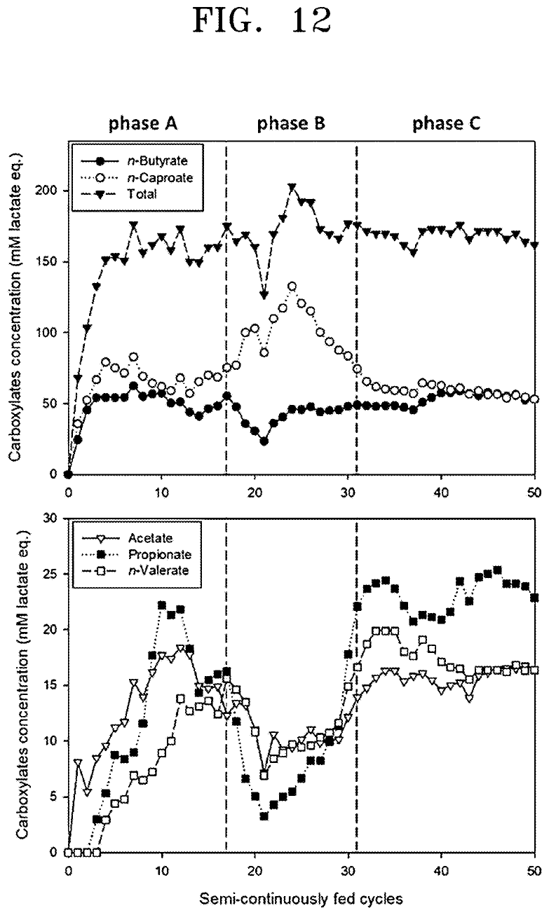

The carboxylate concentration in the effluent during the semi-continuously fed AnMBR operation is shown in FIG. 12. In the 17th cycle, the operation of the circulating water bath was stopped and the temperature of the reactor was lowered to 25° C. The concentration of carboxylate in the effluent increased significantly after the stop, based on the results, the operation period was divided into three phases: Initial operation period (phase A, 0 to 17th cycle), cooling shock period (phase B, 17th to 31st cycle), and recovery period (phase C, 31st to 50th cycle). In phase A, the concentration of n-caproate was about 20 mM to 25 mM after the initial three cycles, and the concentration of n-butyrate was also relatively constant, ranging from 22 mM to 27 mM. However, the sum of the concentrations of propionate and n-valerate, which are carboxylates with odd carbon numbers, increased during the initial 12 cycles. In the 17th cycle, the circulating water bath was stopped, and the culture temperature dropped to 25° C. As a result, the consumption rate of L-lactate dropped sharply, and it was not exhausted even after 24 hours. It took 72 hours for L-lactate to be completely consumed, and semi-continuous operation resumed after L-lactate was completely depleted. After the 17th cycle, the concentration of n-caproate increased rapidly, reaching 44.2 mM by the 24th cycle, while the concentrations of propionate and n-valerate decreased to 3.25 mM and 3.45 mM, respectively, by the 21st cycle.

However, the concentration of n-caproate subsequently decreased while the concentrations of propionate and n-valerate increased, and eventually the productivity of n-caproate in phase C was lower than that in phase A, and the productivity of carboxylates with odd carbon numbers was higher. Specifically, during the SBR operation, carboxylates with odd carbon numbers were produced in negligible concentrations (in other words, less than 0.5 mM), but under the semi-continuous feeding system, the concentrations of propionate and n-valerate reached 17.8 mM and 7.45 mM, respectively, in the 30th cycle. It is known that bacteria that consume L-lactate to produce propionate may be inhibited by adjusting the pH condition to 5.0 or less. Therefore, the pH was continuously adjusted during cycles 31 to 40, resulting in a 5 mM decrease in the concentrations of propionate and n-valerate. However, the concentration of n-caproate did not show a significant difference, indicating that adjusting the pH below 5.0 in the AnMBR system is not a suitable way to increase the selectivity of n-caproate.

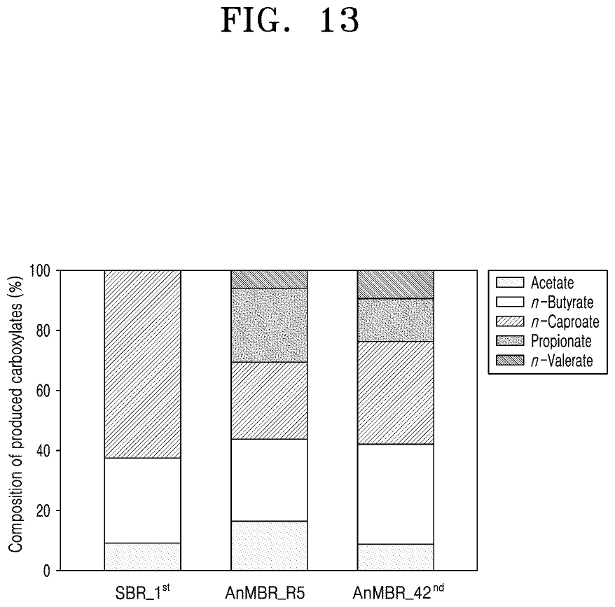

On the other hand, odd carbon number carboxylates were identified in the 3rd DBTL cycle when the concentration of total carboxylate was high. In the semi-continuously fed AnMBR system, the theoretical total carboxylate concentration was 175 mM, which was much higher than the production of odd carbon number carboxylates in the 3rd DBTL cycle. Therefore, to determine the effect of total n-carboxylate concentration on n-caproate production, the entire microbiome was cultured in the R5 experimental setup of the 3rd DBTL cycle. For this purpose, 150 mL of media was collected from the 42nd cycle, centrifuged, and resuspended in a modified basal media containing 50 mM and 10 mM of L-lactate and n-butyrate and cultured. As a result, when the microbiome obtained from AnMBR was cultured in the R5 experimental setup (AnMBR_R5), the specificity of n-caproate was 25.4%, which was lower than when cultured in the effluent of the 42nd cycle of AnMBR (AnMBR_42^{nd}) (FIG. 13). Therefore, based on the above results, it can be seen that the total concentration of carboxylate produced does not affect the production of odd carbon number carboxylate.

Propionate may be produced through the acrylic acid pathway in a reactor supplied with lactate, and propionate may be produced from the residual L-lactate in the reactor. In the case of the semi-continuous feeding system, there may be times of high concentration of residual L-lactate in the reactor due to the substrate being injected at once, and propionate may be actively produced during the period. Therefore, in the following Example 5, the AnMBR was operated in a continuous feeding system to minimize the concentration of residual L-lactate in the media.

### 4.2: Microbial Composition Analysis of Microbiomes Formed After Semi-Continuously Fed AnMBR Operation

To analyze the microbial composition of the microbiome formed after operation of the anaerobic membrane bioreactor with semi-continuous feeding, the following experiments were conducted.

Specifically, microbial analysis was performed as described above in Example 3.2, and during operation of the semi-continuously fed AnMBR, biomass samples were obtained at the 7th and 29th cycles to analyze the composition of the microbiome. The biomass sample of the 7th cycle was obtained from the media, which grew with the attachment of bacteria as the operation continued, and the sample of the 29th cycle was taken from the media, the reactor wall and the hollow-fiber membrane.

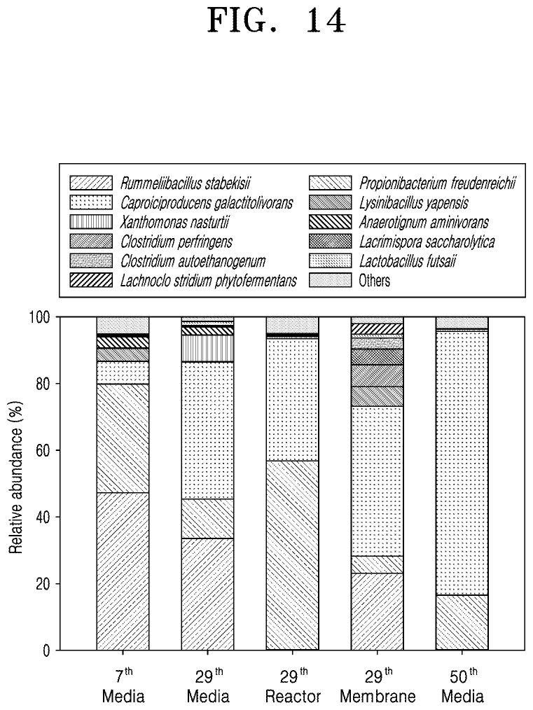

As a result of the above analysis, it was confirmed that the occupancy rates of R. stabekisii, C. galactitolivorans, and P. freudenreichii were high (FIG. 14). Compared to the microbiome analysis performed during SBR operation, R. stabekisii was more dominant, while C. carboxidivorans lost its ecological status, which is a result likely according to prolonged culture. Based on the results, it seems that C. galactitolivorans won the competition with C. carboxidivorans and became the dominant species responsible for the chain extension reaction in the semi-continuously fed AnMBR system.

In addition, the composition of the three dominant species changed during the operation. Specifically, the ratio of C. galactitolivorans, a bacterium that produces n-caproate in the fermentation broth, increased as the operation period lengthened, while R. stabekisii slowly decreased and disappeared by the 50th cycle. C. galactitolivorans is a known n-caproate-producing bacterium that utilizes L-lactate as an electron donor in the chain extension reaction. Therefore, the culture conditions indicate that n-caproate-producing strains may be dominant in the bioreactor system.

### Example 5: Establishment of Operating Conditions for Continuously Fed Anaerobic Membrane Bioreactor for Forming n-Caproate Producing Microbiome

### 5.1: Operation of a Semi-Continuously Fed AnMBR Followed by Operation of a Continuously Fed AnMBR

As confirmed in Example 4 above, the L-lactate provided during an operation of the semi-continuously fed AnMBR was consumed in two competing reactions, namely the chain extension reaction and the acrylic acid reaction, and the yield and selectivity of n-caproate seemed to decrease due to the competing reactions. Therefore, a semi-continuously fed operation followed by a continuously fed operation was performed to determine the production efficiency of n-caproate.

Specifically, after 50 cycles of reaction in semi-continuously fed operation, the bioreactor broth was diluted with modified basic media without substrates (L-lactate and n-butyrate) to reduce the concentration of carboxylate in the bioreactor broth. After dilution of the bioreactor broth, the AnMBR was continuously fed with the same media used in the semi-continuous feeding system (modified basic media including trace metal solution, vitamin solution, yeast extract at 1.25 g/L, 125 mM L-lactate and 25 mM n-butyrate). The reactor was named AnMBR_StoC. During the operation period, the hydraulic retention time was set to 2.5 days to make the organic loading rate (OLR) equal to the loading rate in the semi-continuously feeding period. The circulating water bath was used and the temperature was set to 35° C. The headspace gas of the reactor was pumped into the 1 M NaOH solution using a gas-lift diaphragm pump. A hollow-fiber hydrophilic membrane module was used to filter the effluent and culture the biomass in the reactor. Using an automatic pH controller connected to a liquid-lift diaphragm pump for adding 3.5% HCl solution, the pH was set to 5.5.

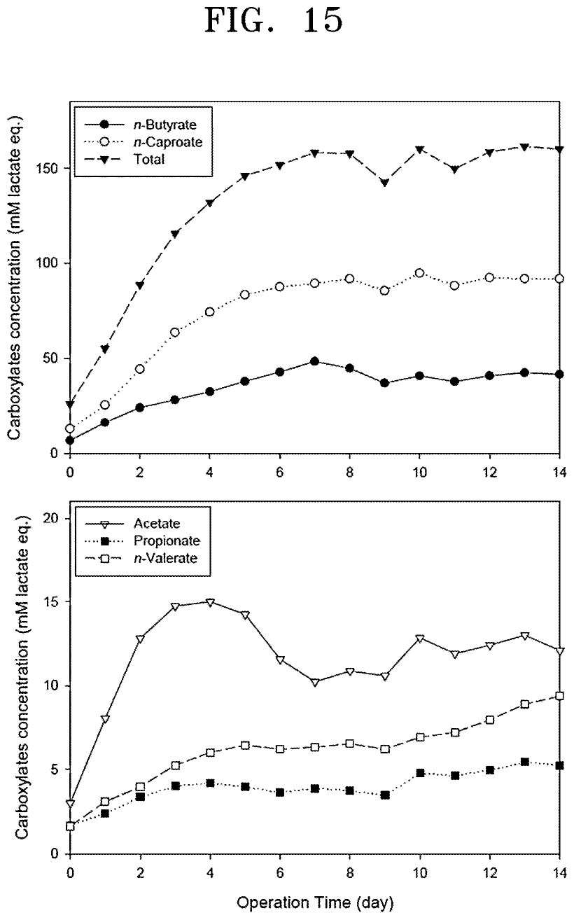

As a result of the operation, the operation was started with a continuous feeding system to keep the concentration of L-lactate at a low level. Since the microbiome had already consumed a lot of L-lactate, L-lactate was no longer detected in the reactor broth. FIG. 15 shows the results of the continuously fed AnMBR (AnMBR_StoC). After 7 days of operation, the concentrations of the main fermentation products (N-butyrate and N-caproate) remained at a constant level (Table 4).

TABLE 4

| | Semi-continuously | |
|---|---|---|
| | fed AnMBR | Continuously |
| | (Semi-continuously | fed AnMBR |
| | fed AnMBR) | (AnMBR_{—StoC}) |
| Operation period | 32 to 50 cycles | 8 to 14 days |
| (Operation | (phase C) | |
| period) | | |
| n-Butyrate | 53.0 ± 4.3 (31.4%) | 41.7 ± 3.7 (26.7%) |
| n-Caproate | 59.3 ± 3.8 (35.2%) | 90.7 ± 2.9 (58.1%) |
| Acetate | 15.7 ± 0.8 (9.3%) | 11.7 ± 1.1 (7.5%) |
| Propionate | 23.2 ± 1.5 (13.8%) | 4.5 ± 0.7 (2.9%) |
| n-Valerate | 17.5 ± 1.4 (10.4%) | 7.4 ± 1.2 (4.8%) |
| Total | 168.8 ± 4.7 | 156.1 ± 6.5 |

During phase C, the semi-continuous operation period, the concentrations of n-butyrate and n-caproate were 53.0 mM±4.3 mM and 59.3 mM±3.8 mM (lactate equivalent), respectively. The specificity of n-caproate could be increased from 35.2% to 58.1% by changing the supplying method, but the specificity of other carboxylates decreased (Table 4). Therefore, the composition of P. freudenreichii, a propionate fermenting bacteria, is expected to decrease, while C. galactitolivorans may dominate in competition for L-lactate, an electron donor.

On the other hand, by changing the supplying scheme from semi-continuous to continuous, the specificity for n-caproate was increased, and n-caproate and n-butyrate were stably produced from the supplied media. However, despite this change, the concentration of carboxylates with odd carbon numbers gradually increased during the operation period. Based on the results, it may be concluded that the semi-continuously feding operation period led to the enrichment of odd-carbon number carboxylate-producing strains, and it is difficult to reconstitute this microbiome.

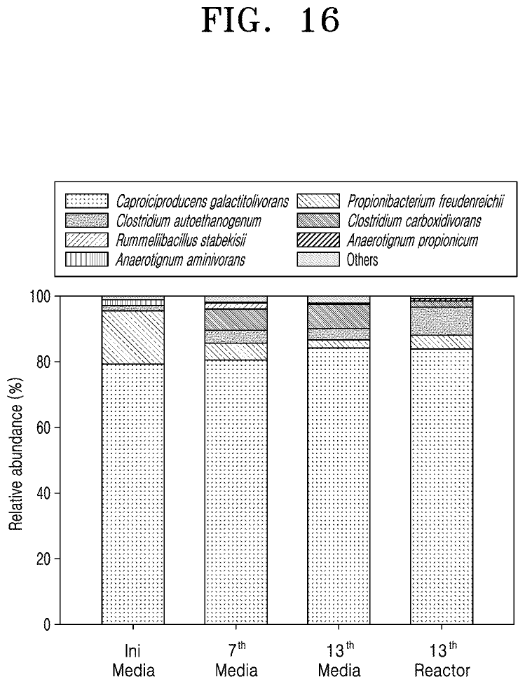

Next, it was found that the selectivity for n-caproate-producing microorganisms could be increased by changing from a semi-continuous to a continuous feeding method. Specifically, during operation of the semi-continuously fed AnMBR, L-lactate was present at high concentrations (L-lactate eutrophic state) immediately after the supply media was injected, and competition for L-lactate was relatively mild. In comparison, during the operation period of the continuously fed AnMBR, the L-lactate concentration was below the detection limit, and the system reached chemostat after 7 days of fermentation (L-lactate oligotrophic state). FIG. 16 shows the composition of the microbiome during AnMBR_StoC operation. The relative abundance of C. galactitolivorans increased significantly by more than 80%, while P. freudenreichii decreased to less than 5%. Bacterial species known to consume CO₂ and H₂ were abundant in the reactor, and the relative abundance of the homoacetogenic bacteria Clostridium autoethanogenum and C.carboxidivorans reached 11.1% on the 13th day of the operation period of AnMBR StoC.

### 5.2: Operates as Continuously Fed AnMBR from Beginning

To prevent dominance of odd-carbon number carboxylate-producing strains, the AnMBR was operated under a continuous feeding system from the beginning, with a shaped microbiome that had undergone three cycles in the SBR used as an inoculum. The AnMBR was named AnMBR Cont.

Specifically, fermentation was carried out in a 2.5 L double-walled reactor. To obtain the shaped microbiome from anaerobic digestive sludge, the reactor was operated under batch culture conditions. After complete depletion of L-lactate, the reactor broth was centrifuged and resuspended in the same media. The circulating water bath was used and the temperature was set to 35° C. After three repeated operations of the batch reactor, the reactor was operated under a continuous feeding regime. The headspace gas in the reactor was pumped into a 1 M NaOH solution using a gas-lift diaphragm pump. The pH was set to 5.5 using an automatic pH controller connected to a liquid-lift diaphragm pump for adding a 3.5% HCl solution. The effluent was filtered using a hollow-fiber hydrophilic membrane module and the biomass was cultured in the reactor. The influent media was a modified basic media including trace metal solution, vitamin solution, 1.25 g/L yeast extract, 125 mM L-lactate, and 25 mM n-butyrate. The pH was set to 5.2 using NaOH. The hydraulic retention time was set to 2.5 days for the operation period.

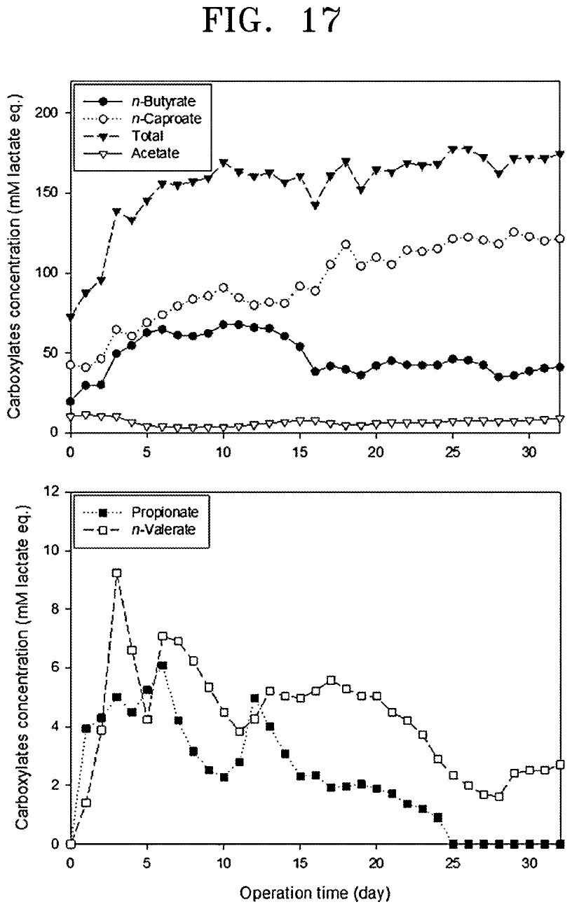

As a result of the above experiment, during the first two weeks of the operation period, the concentration of odd-carbon number carboxylates in the effluent was maintained at about 3 mM (FIG. 17). On the 15th day of the AnMBR operation period, an HCl transfer pump was connected for pH control, after which the concentration of odd-carbon number carboxylates decreased, while the specificity of n-caproate gradually increased. Finally, the concentration of propionate in the AnMBR_cont effluents decreased below the detection limit after the 25th day of operation. On the other hand, the concentration of n-caproate increased steadily and reached 41.9 mM on the 29th day of operation. In addition, the specificity of n-caproate reached 70.5% during the period from day 25 to day 32 (Table 5).

TABLE 5

| | AnMBR_{—StoC} | AnMBR_{—Cont} |
|---|---|---|
| Operation period | 8 to 14 days | 25 to 32 days |
| (Operation | | |
| period) | | |
| n-Butyrate | 41.7 ± 3.7 (26.7%) | 40.7 ± 4.1 (23.6%) |
| n-Caproate | 90.7 ± 2.9 (58.1%) | 121.6 ± 2.2 (70.5%) |
| Acetate | 11.7 ± 1.1 (7.5%) | 8.0 ± 0.6 (4.6%) |
| Propionate | 4.5 ± 0.7 (2.9%) | n.a. |
| n-Valerate | 7.4 ± 1.2 (4.8%) | 2.2 ± 0.4 (1.3%) |
| Total | 156.1 ± 6.5 | 172.5 ± 4.8 |

Considering that it was 35.2% in phase C of the semi-continuously fed AnMBR operation period and 58.1% in the AnMBR_StoC operation period, it may be seen that the reducing power of L-lactate may be focused on the n-caproate production reaction by appropriately changing the experimental conditions and system. The overall carboxylate concentration in the AnMBR_cont effluent was also higher compared to the semi-continuously fed AnMBR and AnMBR_StoC.

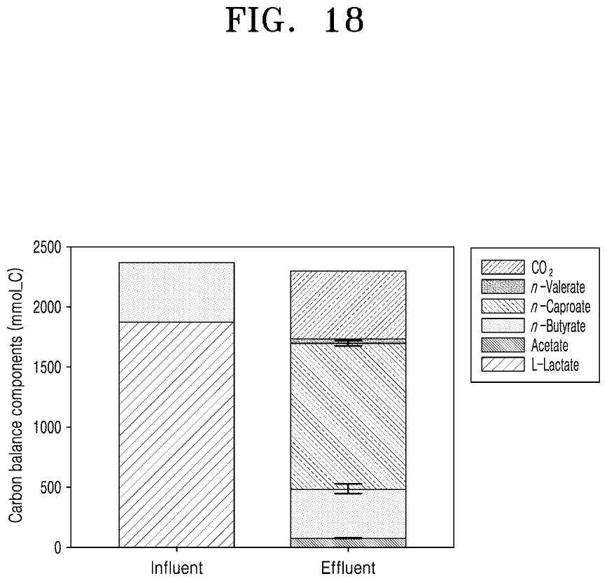

Next, a carbon mass balance analysis was performed to determine the carbon distribution and rearrangement efficiency after AnMBR_cont operation (FIG. 18). Specifically, during the operation period from day 25 to day 32, the carboxylate concentration in the effluent was constant, thus this period was assumed to be the chemostat. The conversion of yeast extract to carboxylates was assumed to be negligible. The carbon in the influent consisted of L-lactate (125 mM) and n-butyrate (25 mM), and after the fermentation reaction in AnMBR_cont, carboxylates and CO₂ were produced. The carboxylate concentration in the effluent was analyzed by the method described above. The produced CO₂ was continuously removed from the headspace using a CO₂ capture module. The CO₂ concentration in the headspace was below the detection limit of the thermal conductivity detector. Therefore, the CO₂ discharged from the fermentation broth into the headspace was assumed to be completely captured by the NaOH solution. The pH change of the NaOH was measured, and the amount of captured CO₂ was calculated based on the acid-base neutralization reaction.

As a result of the above analysis, it was found that 2375 mmol C of carbon in the influent was converted to 1730.5 mmol_C of carbon and 568 mmol_C of CO₂ in the effluent. The carbon was mainly distributed in n-caproate, amounting to 1215.9 mmol_C. The overall carbon to n-caproate conversion rate was 51.2% and the specificity of n-caproate in the effluent was 70.2%. The overall carbon to CO₂ conversion was the second highest at 23.9%. In L-lactate-consuming chain extension bacteria, it is estimated that one out of every three molecules of carbon in L-lactate is converted to CO₂, and 625 mmol_C of carbon is distributed in the form of CO₂. Considering the above assumptions, it is expected that 57 mmol_C of CO₂ will be converted to acetate.

Taking the above results together, It may be seen that an n-caproate producing bioreactor system with high specificity for the target product can be constructed through a system that may maintain optimal conditions to promote dominance of the desired strain in an ecological niche. Furthermore, the n-caproate conversion efficiency of the system of the present disclosure was high enough to neglect the production of odd-carbon number carboxylates during the operation period of 32 days, indicating that the efficiency is excellent.

### 5.2: Microbial Composition Analysis of Microbiomes Formed after Continuously Fed AnMBR Operation

To analyze the microbial composition of the microbiome formed after operation of the anaerobic membrane bioreactor with continuous feeding, the following experiments were conducted.

Specifically, microbial analysis was performed as described above in Example 3.2, and during operation of the continuously fed AnMBR, biomass samples were obtained from the media before operation, on the 7th day of operation, and on the 40th day of operation to analyze the composition of the microbiome.

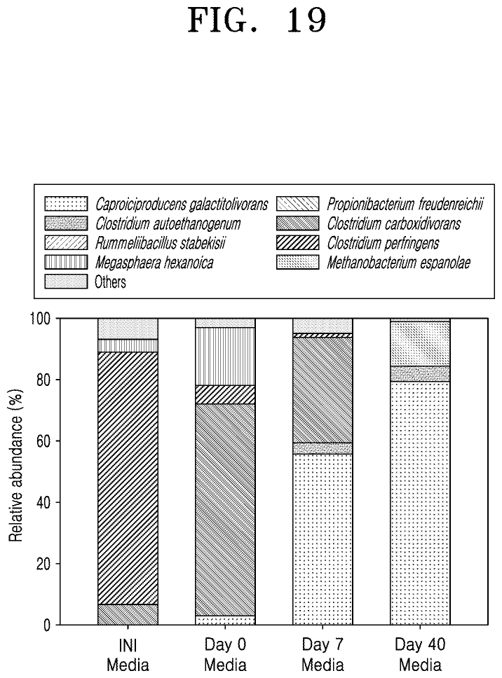

As a result of the above analysis, the relative abundance of the chemolithotrophic homoacetogens decreased by 4.9% on day 40, indicating that this microbiome composition can induce changes in the carboxylate composition in the AnMBR_cont effluent. In addition, it was confirmed that the abundance of C. galactitolivorans strains gradually increased, reaching approximately 80% on the 40th day of operation (FIG. 19).

### Example 6: n-Caproate Production Using n-Caproate Producing Microbiome

To confirm that n-caproate may be produced using milk processing wastewater as a raw material and the microbiome obtained in the embodiment, the following experiments were conducted.

### 6.1: Lactic Acid Fermentation Process

In order to utilize milk processing wastewater containing a high concentration of lactose as a raw material for the n-caproate production platform, a fermentation process to convert lactose into lactate is required, and the following experiments were conducted.

Specifically, expired liquid yogurt (Bulgari's, Namyang Dairy) which was 2 days past its expiration date as an inoculum. The liquid yogurt was found to include microbial species of Streptococcus thermophiles, Lactobacillus acidophilus, Bifidobacterium animalis, Lactobacillus fermentum, and Lactobacillus plantarum. In addition, milk processing wastewater was collected and used from the Pyeongtaek plant of Daily Dairy, and the milk processing wastewater was used as a substrate for lactic acid fermentation. For lactic acid fermentation, a modified basic media including trace metal solution, vitamin solution, 1.25 g/L of yeast extract, 10 mL/L liquid yogurt, and 100 mL/L milk processing wastewater was prepared, and 2 L of culture media was dispensed into a 2.5 L double-walled reactor and cultured. The inoculum, expired liquid yogurt, contained high concentrations of glucose, fructose, and galactose, and was diluted 100 times so that the media included approximately 3 g/L of monosaccharides at the start of fermentation. The circulating water bath was used to set the temperature to 37° C., and an automatic pH controller connected to a liquid-lift diaphragm pump for adding 1 M NaOH solution was used to set the pH to 4.5. The reaction was monitored for 48 hours, and the concentrations of sugars and carboxylates were analyzed using a high-performance liquid chromatography system (DIONEX, California, USA). The concentration of L-lactate reached 50 mM after 24 hours, and 250 mL of fermentation broth was sampled to conduct the n-caproate production experiment.

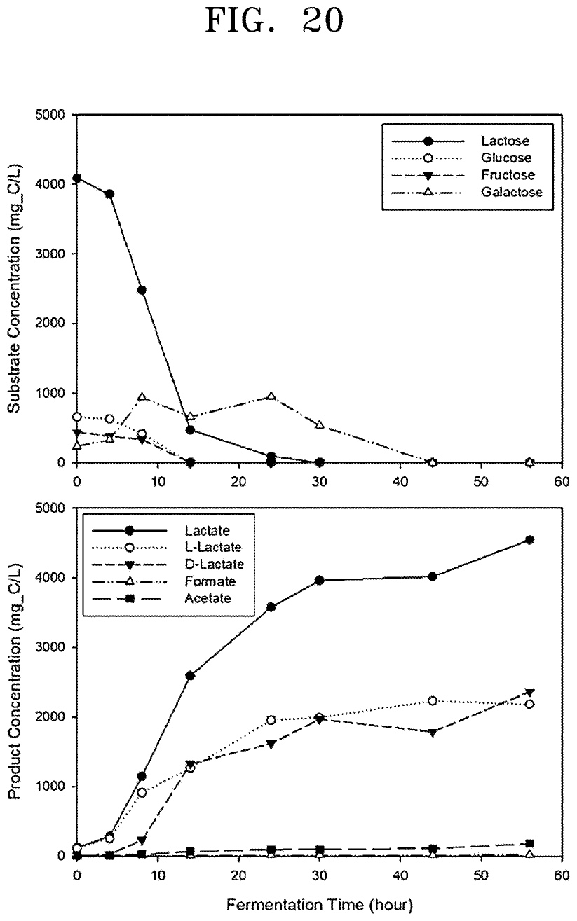

As a result, it was confirmed that once fermentation begins, lactose, glucose, and fructose were rapidly degraded and lactate was produced (Table 6, FIG. 20). Specifically, glucose and fructose were consumed in 14 hours or less, and lactose, the main component of milk processing wastewater, was completely consumed in 30 hours. However, galactose, another product of the lactose hydrolysis reaction, accumulated in the fermentation broth, specifically, during the first 8 hours of fermentation, galactose accumulated in the fermentation broth and its concentration reached 2370 mg/L in 24 hours, but after lactose was depleted, galactose was also rapidly consumed and completely disappeared in 42 hours or less. On the other hand, it is known that the use of galactose may be inhibited in the presence of glucose, thus the interaction between substrates should be considered to optimize the utilization efficiency of milk processing wastewater.

TABLE 6
Milk processing wastewater

| | Lactose | Glucose | Lactose | Glucose | TOC |
|---|---|---|---|---|---|
| pH | (g/L) | (g/L) | (g_C/L) | (g_C/L) | (g_C/L) |
| 6.6 | 105.0 | 0.34 | 44.2 | 0.14 | 44.7 ± 0.9 |

Lactic acid fermentation media

| | Lactose | Glucose | Fructose | Galactose | lactate |
|---|---|---|---|---|---|
| pH | (mg_C/L) | (mg_C/L) | (mg_C/L) | (mg_C/L) | (mg_C/L) |
| 4.5 | 4086.5 | 660.3 | 438.7 | 233.4 | 127.6 |

In addition, lactate, the target product of the fermentation process, was actively produced during the fermentation process, and its final concentration reached 11370 mg/L (4543 mg_C/L). Considering that the initial sugar concentration was 5410 mg_C/L, the conversion rate from sugar to lactate was 87.1% (calculated based on mg_C/L). The concentration of acetate, a heterofermentative LAB active product, was 457.8 mg/L, showing a selectivity of 4.0%. Thus, it may be seen that homofermentative bacteria won the ecological niche and have high sugar-lactate selectivity. In addition, L-lactate was the dominant fermentation product during the first 8 hours of fermentation, resulting in an L-lactate/D-lactate ratio of 3.90, and the ratio was founded to be decreased to 1.21 during the next 24 hours of fermentation (Table 6, FIG. 20).

### 6.2: n-Caproate Production Process

To produce n-caproate using the lactic acid fermentation broth obtained in Example 6.1 above, the following experiments were conducted.

Specifically, the fermentation broth was obtained when the concentration of L-lactate reached 50 mM by fermentation for about 24 hours in the lactic acid fermentation process of Example 6.1 above, and was filtered using centrifugation and a 0.45 um syringe GHP filter. To obtain the n-caproate producing microbiome, 150 mL of fermentation broth was collected from AnMBR_cont in Example 4 above, centrifuged, and resuspended in lactic acid fermentation broth. In addition, the experimental setup used in the 3rd DBTL cycle of Example 2 above was used as the reaction vessel.

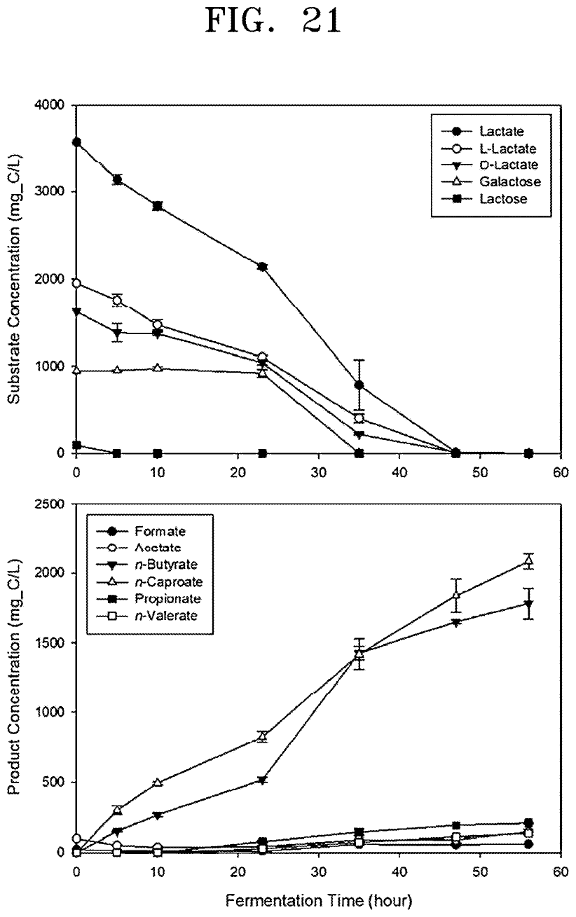

As a result, the total lactate concentration (sum of L-lactate and D-lactate concentrations) before performing the n-caproate production process was 99.3 mM, and during the operation period of the batch culture reactor, both L-lactate and D-lactate were completely consumed after 48 hours of culture, which indicates that the bacteria in the microbiome obtained through the present disclosure possess both L-lactate dehydrogenase and D-lactate dehydrogenase. Furthermore, galactose degradation required an acclimatization period, whereas lactose was completely consumed in 5 hours or less. The galactose concentration remained constant during the initial 23 hours, but was rapidly consumed during the 23 to 35 hours of the operation period (FIG. 21).

Next, the concentration changes of carboxylates were checked in the processes, and n-caproate was actively produced during the 56th hour of the operation period, reaching 28.91 mM (86.73 mM lactate equivalent, 2082 mg_C/L). n-Butyrate was also actively produced, reaching a concentration of 37.1 mM (74.2 mM lactate equivalent, 1780 mg_C/L), especially the production of n-butyrate increased from 23 to 35 hours, when the galactose concentration decreased rapidly. Therefore, it may be seen that the degradation of galactose may lead to production of n-butyrate.

On the other hand, C. galactitolivorans included in the n-caproate producing microbiome is known to utilize galactose, and the sum of the concentrations of n-caproate and n-butyrate produced (3862 mg_C/L) was higher than the initial lactate concentration (3574 mg_C/L), suggesting that galactose may also contribute to n-caproate production.

The description of the present disclosure described above is for illustrative purposes, and those skilled in the art will understand that the present disclosure can be easily modified into other specific forms without changing the technical idea or essential features of the present disclosure. Therefore, the embodiments described above should be understood in all respects as illustrative and not restrictive.

1. A method of preparing a caproate-producing microbiome, comprising:
a process of inoculating an anaerobic microbiome in a media composition comprising lactate; and
a process of culturing the media composition.

2. The method of claim 1, wherein the process of culturing comprises a process of making a caproate-producing strain dominant within the anaerobic microbiome.

3. The method of claim 2, wherein the caproate-producing strain comprises one or more selected from the group consisting of Ruminococcacea spp., Ruminococcaceae bacterium CPB6, Clostridium kluyveri, Megasphaera elsdenii, Megasphaera hexanoica, Caproiciproducens genus, Clostridium carboxidivorans, and Caproiciproducens galactitolivorans.

4. The method of claim 1, wherein the process of culturing comprises culling a propionate-producing strain within the anaerobic microbiome.

5. The method of claim 4, wherein the propionate-producing strain comprises one or more selected from the group consisting of the genus Propionibacterium comprising Propionibacterium freudenreichii, Propionibacterium acidipropionici, Propionibacterium jensenii, Propionibacterium thoenii, etc., Veillonella gazogenes, Veillonella criceti, Veillonella alcalescens, Veillonella parvula, Clostridium homopropionicum, Bacteroides spp. and Fusobacterium necrophorum.

6. The method of claim 1, wherein the lactate is comprised in the media composition at a concentration of 10 mM to 150 mM.

7. The method of claim 1, wherein the media composition additionally comprises butyrate.

8. The method of claim 7, wherein the butyrate is comprised in the media composition at a concentration of 1 mM to 50 mM.

9. The method of claim 7, wherein the lactate and butyrate are comprised in the media composition at a ratio of 1:1 to 20:1.

10. The method of claim 1, wherein a headspace gas in the process of culturing includes hydrogen.

11. The method of claim 1, wherein the process of culturing additionally comprises removing carbon dioxide from a headspace.

12. The method of claim 1, wherein in the process of culturing, pH of the media composition is 5 to 6.

13. The method of claim 1, wherein the process of culturing is culturing in batch culture, continuous culture, semi-continuous culture, or a combination thereof.

14. The method of claim 13, wherein the batch culture is performed with 1 to 10 repetitions.

15. The method of claim 13, wherein the semi-continuous culture is performed at a temperature condition of 20 to 30° C.

16. A method of producing caproate, comprising:
a process of inoculating an anaerobic microbiome in a media composition comprising lactate; and
a process of culturing the media composition.

17. The method of claim 16, wherein the process of culturing comprises a process of making a caproate-producing strain dominant.

18. A method of producing caproate, comprising:
a process of inoculating an organic waste resource comprising lactate with a caproate-producing microbiome prepared by the method of claim 1; and
a process of culturing the organic waste resource.


SEQUENCE LISTING (2 sequences)

SEQ ID NO: 1 - DNA, 17 bp, Artificial Sequence
  Description of Artificial Sequence Synthetic oligonucleotide
  modified_base (9)..(9): n is a, c, g, or t
cctacgggnggcwgcag

SEQ ID NO: 2 - DNA, 21 bp, Artificial Sequence
  Description of Artificial Sequence Synthetic oligonucleotide
gactachvgggtatctaatcc